(19) United States
(12) Patent Application Publication
(10) Pub. No.: US 2014/0271573 A1
(43) Pub. Date: Sep. 18, 2014

(54) PHARMACO-CELLULAR THERAPEUTIC METHOD FOR THE TREATMENT OF MUSCULAR DYSTROPHIES

(71) Applicant: PONTIFICIA UNIVERSIDAD CATÓLICA DE CHILE, Santiago (CL)

(72) Inventors: Enrique Brandan, Santiago (CL); Daniel Cabrera, Santiago (CL); Jaime GUTIERREZ, Santiago (CL); Gabriela Morales, Santiago (CL); Claudio CABELLO-VERRUGIO, Santiago (CL); Juan Hancke, Santiago (CL)

(73) Assignee: PONTIFICIA UNIVERSIDAD CATÓLICA DE CHILE, Santiago (CL)

(21) Appl. No.: 14/213,403

(22) Filed: Mar. 14, 2014

Related U.S. Application Data
(60) Provisional application No. 61/783,229, filed on Mar. 14, 2013.

(51) Int. Cl. A61K 36/19; A61K 31/365; A61K 35/34

(52) U.S. Cl. CPC A61K 36/19; A61K 35/34; A61K 31/365
USPC 424/93.7

(57) ABSTRACT

A method of treating a muscular dystrophy disease in a patient includes administering an effective amount of a botanical drug isolated from Andrographis paniculata in combination with cell therapy. The method improves skeletal muscle performance.

### FIELD OF THE INVENTION

The current invention is related to a method for treating muscular dystrophies using a combination of therapies which found to be more effective than those therapies applied individually.

Specifically, the current invention refers to the use of a botanical drug isolated from Andrographis paniculata together with a cell therapy for the treatment of Muscular Dystrophies, e.g., Duchenne muscular dystrophy (DMD).

In this invention, we demonstrated the impact of andrographolide in the progression of dystrophic diseases, evaluating fibrosis induction, muscle strength and finally we demonstrated that andrographolide generates a propitious niche to increase stem cell therapy engraftment.

### BACKGROUND OF THE INVENTION

Muscular Dystrophies are a group of muscular genetic diseases. The most severe is Duchenne muscular dystrophy (DMD). DMD is an X-linked recessive disorder that affects 1 of 3500 birth, for which there is no effective therapy (Kapsa et al., 2003). It is caused by the absence of dystrophin, a cytoskeletal protein that anchors the muscle fiber to the extracellular matrix (ECM). The absence of this protein increases the susceptibility of muscle fiber rupture caused by the continuous cycles of contraction and relaxation (Allen and Whitehead, 2011; Blau et al., 1983). Thus, children with this condition gradually and progressively lose muscle strength, requiring the use of a wheel chair since age 10 and dying in the late second or early third decade of life by to cardio-respiratory arrest due to severe muscle damage in the heart and diaphragm muscles. One cause of this damage and loss of muscle function is the appearance of fibrosis, which is characterized by excessive accumulation of ECM replacing muscle tissue by connective tissue, dramatically affecting the fibers environment and therefore normal muscle physiology.

Some DMD pathological features are myofiber atrophy, fatty degeneration, necrosis and fibrosis but only fibrosis has been correlated through clinical studies with poor motor outcome gauged by muscle strength and age at loss of ambulation (Desguerre et al., 2009). This finding supports the notion that fibrosis directly contributes to progressive muscle dysfunction and the lethal phenotype of DMD.

Fibrosis is defined pathologically as inappropriate repair by connective tissue and is characterized by a loss of normal tissue architecture by dense, homogeneous, and increasingly stable ECM components such as collagens and fibronectin (which can impair tissue function). The process leads to progressive distortion of tissue architecture with consequent dysfunction and ultimate failure of fibrotic organs (Varga et al., 2005; Wynn, 2008). Therefore, finding new drugs and therapies with anti-fibrotic effects is crucial in the field.

Glucocorticoids are the first line therapy in the treatment of DMD, which retards the use of wheelchairs for around 2 to 4 years, but involves troublesome and severe side effects. The most common assumption is that glucocorticoid treatment of dystrophy alleviates the dystrophic process primarily through immune-suppression and reduction of inflammation. However, vertebral fractures are a well-known complication associated with steroid use and should be aggressively treated with bisphosphonate therapy (Verma et al., 2010).

Andrographolide, a bicyclic diterpenoid lactone, is the major constituent of Andrographis paniculata, a plant indigenous to Southeast Asian countries that has been used as an official herbal medicine in China for many years (Shen et al., 2002). Traditionally it is used for the treatment of cold, fever, laryngitis and infection in many Asian countries. Extract of the plant is reported to possess immunological, antibacterial, anti-inflammatory, antithrombotic, hepatoprotective, anti-hypertensive, and anti-diabetic activities (Akbar, 2011). It has been reported to be particularly efficient at regulating immune responses (Calabrese et al., 2000; Rajagopal et al., 2003) and present anti-inflammatory properties by reducing the generation of reactive oxygen species in human neutrophils (Shen et al., 2002). It has been shown that andrographolide not only regulates inflammation, but also regulates fibrosis in chronic liver and kidney diseases. Mechanistically, andrographolide forms a covalent adduct with NF-kappaB, thus blocking the binding of NF-kappaB oligonucleotide to nuclear proteins (Xia et al., 2004). Remarkably, NF-kappaB is an important transcription factor involved in the progression of dystrophic diseases (Acharyya et al., 2007).

However, dystrophic disorders such as DMD have genetic origins, only way to restore the gene expression is through Gene and/or Cell therapies. Nevertheless, these therapies represent a major challenge, since muscle is the most abundant tissue in the body and more over fibrosis reduce the efficacy of these approaches (Zhou and Lu, 2010). Therefore, even if current trials are successful, they are unlikely to elicit a significant benefit when extended to people at more advanced stages of the disease.

Genetic and stem cells therapies have shown promising results diminishing the severity of the disease. Grafted stem cells or muscle progenitors partially restore dystrophin expression, significantly reducing muscle damage and restoring function. One problem associated with these therapies is the low tissue colonization efficiency by the stem/progenitors muscle cells by the presence of an important physical barrier formed by the excessive connective tissue (fibrotic) present in the dystrophic muscle, which impedes the efficient migration and colonization of these stem/progenitor cells (Gargioli et al., 2008). Therefore, understanding cellular and molecular mechanisms underlying muscle fibrogenesis associated with dystrophin deficiency is critical to the development of effective anti-fibrotic therapies for DMD.

We have shown that the use of andrographolide, significantly reduce muscle associated fibrosis, increasing the efficiency of the cell therapy in an animal model of DMD, the mdx mouse. Andrographolide already has been used in other diseases with no or little adverse side effects.

The proposed invention consists in a method for enhancing the efficiency of cell therapy using the natural compound (andrographolide) in fibrotic tissues. This type of strategy is completely new, since a method or an effective therapy for the treatment of DMD is unavailable.

### STATE OF THE ART

DMD is the most common genetic muscle disease. Although gene therapy and cell therapy may ultimately provide a cure for DMD, currently the disease is devastating, with no effective therapies. Recent studies have demonstrated that ameliorating muscle fibrosis may represent a viable therapeutic approach for DMD (Zhou and Lu, 2010). However, the etiology of the disease remains, since DMD is a genetic disorder.

### Therapies for Duchenne Muscular Dystrophies

Mendell et al. discuss different emerged therapeutic strategies that have been used in pre-clinical and clinical settings. Most attractive are molecular-based therapies that can express the missing dystrophin protein (Mendell et al., 2010). However, it has been so difficult bring these therapies to clinical assays, because dystrophin is a big protein and moreover a large gene, then find good vectors to deliver the gene is a trouble. The efficacy of these approaches is very low. The size of the dystrophin gene makes it difficult to work with in gene therapy. Thus smaller genes, micro or mini-dystrophin, have been developed, which can be inserted into a vector. The most suitable vector found so far is a virus associated with the adenovirus, a non-pathogenic parvovirus, but it has been shown to cause an immunological response. In order to assess the response, mdx mice dys-/dys- have been created, and there is evidence that when the gene is injected, the dystrophin is partially expressed and muscular strength is improved. However, in preliminary studies on humans, 90 days after treatment initiation the gene expression was lost (Arechavala-Gomeza et al., 2010; Mendell et al., 2010). These results suggest that cellular immunity inhibits the success of this therapy.

Other approaches include increasing the strength of muscles (myostatin inhibitors), reducing muscle fibrosis and decreasing oxidative stress. Additional targets include inhibiting NF-κB to reduce inflammation or promoting skeletal muscle blood flow and muscle contractility using phosphodiesterase inhibitors or nitric oxide (NO) donors. Nonetheless, these approaches only control the symptoms but not the primary cause of DMD dystrophies that is the absence of dystrophin gene expression, therefore the disease is less severe but is not healed.

Mendellet. al. have used small molecules for exon skipping and mutation suppression and gene transfer to replace or provide surrogate genes as tools for molecular-based approaches for the treatment of muscular dystrophies. Exon skipping is targeted at the pre-mRNA level allowing one or more exons to be omitted to restore the reading frame. In DMD, clinical trials have been performed with two different oligomers, a 2′O-methyl-ribo-oligonucleoside-phosphorothioate (2′OMe) and a phosphorodiamidatemorpholino (PMO). Both have demonstrated early evidence of efficacy (Mendell et al., 2012). A disadvantage of this drug is that the effect is only transitory and limited to the time in which this antisense oligonucleotide remains in the tissue.

Another molecular approach involves suppression of stop codons to promote read through of the DMD gene (Pichavant et al., 2011). In cell cultures, gentamicin interacts with the 40S ribosomal subunit in the transcription of RNA, suppressing the termination codons and inserting in its place another amino acid which replaces it. In studies on mdx mice and in humans, gentamicin was capable of producing dystrophin expression in muscle fibers at 20% of normal levels (Pichavant et al., 2011). In studies on mdx mice and humans, gentamicin was capable of producing dystrophin expression in muscle fibers at 20% of normal levels (Pichavant et al., 2011). However, studies on DMD patients remain controversial. In fact one of them showed a beneficial effect of the muscle strength and a re-expression of dystrophin in muscles, while in another study on 12 DMD patients performed over a six-month period, dystrophin expression was detected in only 6 of the 12 patients, and no clinical benefits were observed (Malik et al., 2010; Wagner et al., 2001).

### Andrographolide and Fibrotic Disorders

There is some evidence that supports the use of andrographolide as an anti-fibrotic compound. For example, Lee M J et. al. studied the anti-diabetic nephropathy effect of diterpene lactones andrographolide (AP1) and 14-deoxy-11,12-didehydroandrographolide (AP2) from Andrographis paniculata (Lee et al., 2010a). They suggested that addition of compounds AP1 or AP2 reduces the phenotypes indicating diabetic nephropathy in MES-13 cells. The compound AP2 showed potent activity than AP1 in the reduction of apoptosis marker caspase-3, fibrosis marker TGF-β1, and PAI-1. Furthermore, AP1 and AP2 do not have antioxidant ability in a cellular environment; however, addition of AP 1 and AP2 reduced intracellular oxidative states in high glucose cultured MES-13 cells. Lee T Y, et. al. have identified andrographolide as a potent protector against cholestasis-induced apoptosis in vivo. Its anti-apoptotic action largely relies on the inhibition of the oxidative stress pathway (Lee et al., 2010b). All this evidences support the idea that andrographolide can inhibit inflammation and fibrosis in organ such as kidney, lung and liver under damage conditions. However, there is no evidence of these results in skeletal muscle and even less in muscular dystrophies.

Patent application N° US 2011/0224128 (Whalen et. al., 2011), called “methods and compositions for treatment of muscular dystrophy” propose combinations of compounds having stimulatory activity toward an alpha 7 integrin promoter element or an inhibitory activity toward NF-kappaB mediated gene activation to treat muscular dystrophies, one of the drugs listed is andrographolide. However, they don't show direct experimental evidence of the use of this drug, and more important is that they don't show or propose the use of andrographolide in combination with cell or molecular therapies to treat muscular dystrophies.

In summary, andrographolide is an interesting drug with anti-inflammatory and anti-fibrotic activities showed in organs such as lung, liver and kidney, however there are not evidences of these activities in skeletal muscle diseases. Is clear that anti-fibrotic and anti-inflammatory therapies decrease or retard symptoms in muscular dystrophies, nevertheless no one of these therapies has the ability to restore the gene expression. A plethora of different strategies have been used to restore the dystrophin expression, nonetheless no one of them has proved fully effective.

There are not previous evidences testing methods that work with anti-fibrotic or anti-inflammatory drugs and gene or molecular therapies working together. This invention proposes a method using the botanical drug andrographolide or extracts with high content of andrographolide together with cell therapy. The results of this invention show an interesting and an unexpected synergic effect of these approaches, since the effect of the combined therapy is better than the sum of both.

### BRIEF DESCRIPTION OF THE DRAWINGS

FIG. 1. Andrographolide inhibits fibrosis induction by TGF-β1 in vitro. A) Myoblasts C2C12 were incubated with 10 ng/ml TGF-β1 and 50μ Mandrographolide for 6 hrs. to evaluate CTGF expression by northern blot analysis. The ribosomal subunits 28 and 18s were evaluated as loading control. B) Myoblasts C2C12 were incubated with 10 ng/ml TGF-β1 and 50μ Mandrographolide for 24 hrs. to determine the levels of fibronectin (FN) and collagen type III (Col III). As loading control was evaluated the levels of tubulin (Tub).

FIG. 2. Andrographolide reduced expression and activity of the pro-fibrotic cytokine, TGF-β1, in mdx mice. To augment the extent of muscle fibrosis, 3 months old mdx mice were subjected to an exercise protocol for 3 months. Along this period one group was treated with 1 mg/kg andrographolide or vehicle (i.p. injections 3 time per week, 4 animal per group). A) TGF-β1 mRNA levels, an important pro-fibrotic cytokine, were determined in tibialis anterior muscle from Wild type (WT), Vehicle-Treated mdx and andrographolide-treated mdx mice by RT-qPCR using GAPDH as reference gene. Values correspond to the mean of dCT value±SD of three independent experiments, using four mice for each experimental condition and normalized to WT levels (*, P<0.05 relative to WT mice; #, P<0.05 relative to Vehicle-treated mdx mice). B) Detection of pSmad-2 (an intracellular TGF-β1 mediator) through indirect immunofluorescence analysis in cryosections of tibialis anterior muscle from Vehicle-treated and andrographolide-treated mdx mice. Bar corresponds to 200 μm.

FIG. 3. Andrographolide modulates CTGF action in vivo. A) To augment the extent of muscle fibrosis, 3 months old mdx mice were subjected to an exercise protocol for 3 months. Along this period one group was treated with 1 mg/kg andrographolide or vehicle (i.p. injections 3 time per week, 4 animal per group). CTGF mRNA levels, a downstream TGF-β1 pro-fibrotic mediator) were determined in tibialis anterior muscle from Wild type (WT), Vehicle-Treated mdx and andrographolide-treated mdx mice by RT-qPCR using GAPDH as reference gene. Values correspond to the mean of dCT value±SD of three independent experiments, using four mice for each experimental condition and normalized to WT levels (*, P<0.05 relative to WT mice; #, P<0.05 relative to Vehicle-treated mdx mice). B) Detection of macrophages (F4/80 positive cells) through immunohistochemistry analysis in cryosections of tibialis anterior muscle from andrographolide-treated WT mice that overexpress CTGF by adenovirus (adv CTGF) infection.

FIG. 4. Andrographolide reduced skeletal muscle damage in mdx mice. To augment the extent of muscle fibrosis, 3 months old mdx mice were subjected to an exercise protocol for 3 months. Along this period one group was treated with 1 mg/kg andrographolide or vehicle (i.p. injections 3 time per week, 6 animal per group). A) Hematoxylin and eosin staining of tibialis anterior muscles showed striking smaller muscle damaged areas in andrographolide-treated mdx mice as compared with untreated mdx mice (scale bars=200 μm). B) Evans blue dye uptake in tibialis anterior muscle fibers from wild-type (WT), Vehicle-treated mdx and andrographolide-treated mdx mice. Nuclei were labeled with Hoechst. Mice were injected i.p. with 1% Evans blue dye 24 hours before muscle fixation. Bar corresponds to 200 μm. C) Bar graph shows a significant reduction of serum Creatine Kinase (CK) activity in andrographolide-treated mdx mice as compared with Vehicle-treated mdx mice. Values are expressed as mean±SD of three independent experiments, using eight mice for each experimental condition. (*, P<0.05 relative to WT mice; #, P<0.05 relative to Vehicle-treated mdx mice).

FIG. 5. Andrographolide reduced skeletal muscle fibrosis in mdx mice. To augment the extent of muscle fibrosis, 3 months old mdx mice were subjected to an exercise protocol for 3 months. Along this period one group was treated with 1 mg/kg andrographolide or vehicle (i.p. injections 3 time per week, 6 animal per group). A) Detection of collagen I and fibronectin by indirect immunofluorescence analysis in cryosections of tibialis anterior muscles from WT, Vehicle-treated mdx and andrographolide-treated mdx mice. Bar corresponds to 200 μm. B) fibronectin (FN) protein levels were detected by western blot analysis in extracts obtained from tibialis Anterior muscles from WT, Vehicle-treated mdx and andrographolide-treated mdx mice. C) collagen type III (Col III) protein levels were detected by western blot analysis in extracts obtained from tibialis anterior muscles from WT, Vehicle-treated mdx and andrographolide-treated mdx mice. GAPDH protein levels are shown as loading control; molecular weight markers are shown in kDa.

FIG. 6. Andrographolide increased skeletal muscle strength and exercise performance in mdx mice. 3 months old Wild Type (WT) and mdx mice were subjected to an exercise protocol for 3 months to augment the extent of muscle fibrosis. Along this period one group was treated with 1 mg/kg andrographolide or vehicle (i.p. injections 3 time per week, 6 animal per group). A) tibialis anterior muscles from wild-type (WT), Vehicle-treated mdx and andrographolide-treated mdx mice were isolated to evaluate isometric specific force (mN/mm2) at different stimulation frequencies (pulses per second, pps). B) Bar graph showing tetanic specific force. Values are represented as percentage of specific isometric force generated by Wild type muscle (*, P<0.05 relative to WT mice; #, P<0.05 relative to Vehicle-treated mdx mice). C) Bar graph showing twitch force (*, P<0.05 relative to WT mice; #, P<0.05 relative to Vehicle-treated mdx mice). D) Mice were subjected to an exercise challenge into the treadmill at 15 meters/min for 5 minutes and the number of stebacks was counted (*, P<0.05 relative to WT mice; #, P<0.05 relative to Vehicle-treated mdx mice).

FIG. 7. Andrographolide increases cell migration through fibrosis inhibition. A) Migration of DiI-labeled tendon fibroblast cells after injection into the tibialis anterior of 7-month-old mdx mice (3 months old mdx mice exercised for four months). Collagen I is revealed by immunofluorescence. B) Quantitative analysis of the spreading DiI-labeled tendon fibroblast cells was calculated by counting DiI-labeled cells in 200 μm×200 μm squares from three non-serial transverse sections for three mice per group. Note that DiI-labeled cells were distributed more homogenously in andrographolide-treated than Vehicle-treated mdx muscles.

FIG. 8. Muscle stem cell therapy with satellite cells is improved by reducing the muscle fibrosis by the treatment with andrographolide. A) 3 months old mdx mice were subjected to an exercise protocol for 4 months to augment the extent of muscle fibrosis. During this period one group was treated with andrographolide or vehicle (i.p. injections 3 time per week, 6 animal per group). 1 week after the last drug administration (7 month old mice), 500 freshly isolated satellite cells (SC) purified from WT mice were transplanted in both tibialis anterior muscle of each mdx mouse. 4 weeks after the engraftment the number of fibers expressing dystrophin and collagen I were determined by immunofluorescence on cryosections. The images are representative of 2 experimental group with 6 mice per group. B) Quantification of the data obtained in A showing the number of myofibers expressing dystrophin per tibialis anterior muscle in each case. C) The number of satellite cells (PAX-7 positive nuclei) were determined on isolated single muscle fibers from extensor digitoriumlongus muscle (EDL) in each case as indicative of the endogenous SC survival.

### DETAILED DESCRIPTION OF THE INVENTION

### Introduction

Embodiments disclosed in the present specification relate to the use of a botanical drug isolated from Andrographis paniculata in combination with stem cell therapy for the efficient treatment of Muscular Dystrophies, e.g., Duchenne muscular dystrophy (DMD).

DMD is a genetic disorder caused by a mutation in the dystrophin gene. The absence of dystrophin is traduced in progressive muscle damage, fibrosis and muscle weakness. Children with this condition, require the use of a wheel chair since the age of 10, dying in the third decade of life due to the severe muscle damage. The only way to restore the dystrophin expression is through Gene and/or Cell therapies. However, the presence of the fibrotic tissue forms a physical barrier for the efficient delivery of any of these therapeutic strategies.

In one exemplary embodiment, a method uses Andrographolide, which reduce the fibrotic tissue in dystrophic muscles, generating a propitious niche to increase the stem cell therapy efficiency. This strategy is completely new, since methods or effective therapies to treat DMD are unavailable.

### Methods

### Cell Cultures

The skeletal muscle cell line C2C12, obtained from adult mouse leg (American Type Culture Collection), was grown and induced to differentiate, as described (Larrain et al., 1997). Myotubes were treated with 10 ng/ml TGF-β1 and/or 50μ Mandrographolide. Cells were serum-starved and then treated for the indicated times.

### RNA Isolation and Northern Blot Analysis

Total RNA was isolated from cultures as described previously (Brandan et al., 1992). Twenty micrograms of RNA samples were electrophoresed in 1.2% agarose/formaldehyde gels, transferred onto Nytran membranes (Schleicher & Shuell, Dassel, Germany) and hybridized with random primed [³²P]dCTP-labeled cDNA probes for mouse CTGF in hybridization buffer over night at 42 or 65° C., respectively. Hybridized membranes were then washed at 42° C. and exposed to Phosphor Imager and Kodak X-ray films. The cDNA probe for mouse CTGF corresponds to a fragment of 532 bp that was amplified by RT-PCR using the following primers: Forward: 5′-GAG TGG GTG TGT GAC GAG CCC AAG G-3′ and reverse: 5′-ATG TCT CCG TAC ATC TTC CTG TAG T-3′ (Vial et al., 2008).

### Immunoblot Analysis.

For immunoblot analyses, muscles were homogenized in 10 volume Tris-EDTA buffer with 1 mM PMSF as described previously (Morales et al., 2011). Briefly, proteins were determined in aliquots of muscle extracts using the bicinchoninic acid protein assay kit (Pierce, Ill.) using BSA as standard. Aliquots (50-100 μg) were subjected to SDS gel electrophoresis in 8% or 10% polyacrylamide gels, electrophoretically transferred onto PVDF membranes (Schleicher & Schuell) and probed with specific antibodies against fibronectin (Sigma-Aldrich, USA), collagen III (Rockland, USA and GAPDH (Millipore, USA), tubulin (Sigma-Aldrich, USA) and GAPDH (Sigma-Aldrich, USA). All immunoreactions were visualized by enhanced chemiluminescence kit (Pierce, USA). Densitometric analysis and quantification were performed using ImageJ software (NIH, USA) (Cabello-Verrugio et al., 2012).

### Animals and Experimental Exercise.

Control or mdx (12 weeks old) male mice of C57BL/10 ScSn strain were studied. The animals were kept at room temperature with a 24 hour night-day cycle and fed with pellets and water ad libitum. Experimental exercise was performed for running the mice in a treadmill for three times per week, 30 minutes each time at 12 m/min during 3 or 4 months (De Luca et al., 2005; De Luca et al., 2003). Along this time, two experimental groups were designed: those treated with vehicle or andrographolide (1 mg/Kg/day). At the end of the experiment, muscles were dissected and removed under anesthesia, and then the animals were sacrificed. Tissues were rapidly frozen and stored at −80° C. until processing or used to electrophysiological measurement. All protocols were conducted in strict accordance and with the formal approval of the Animal Ethics Committee of the P. Universidad Católica de Chile.

### Serum Creatine Kinase (CK) Measurement.

Mice were anesthetized by isofluorane gas, and blood was obtained from the periorbital vascular plexus directly into microhematocrit tubes (70 μl, Fisher Scientific). Serum was obtained by allowing the blood to clot at room temperature for 30 minutes and then centrifuging at 1,700×g for 10 minutes. Serum CK was measured by enzymatic system (Valtek, Chile) according to the manufacturer instructions) (Osses and Brandan, 2002).

### EBD Uptake.

Animals were injected with Evans Blue dye (1% in PBS) and allowed to stay for 24 hours. Mice were then sacrificed and tibialis anterior muscles were snap frozen in isopentane, then were sectioned in 7 μm cryosections and fixed in 4% paraformaldehyde. Muscle cross sections were visualized under a Nikon Diaphot inverted microscope, equipped for epifluorescence. The percentage of Evans blue dye positive fibers was manually counted in a blinded manner (Straub et al., 1997).

### Immunofluorescence Microscopy.

For immunofluorescence, snap-frozen muscles in thawing isopentane were sectioned and cryosections (7 μm) were fixed in 4% paraformaldehyde, blocked 1 hour in 10% goat serum in PBS, incubated for one hour at room temperature with specific antibodies against fibronectin (Sigma, USA), collagen I (Chemicon, USA), F4/80 (abcam, USA), p-Smad2 (abcam, USA) and dystrophin (Santa Cruz, USA). As secondary antibody FITC-conjugated goat anti rabbit IgG and rabbit anti mouse IgG (Thermom USA) were used. For monoclonal anti mouse antibodies all the incubations were made with mouse IgG-blocking solution from the M.O.M. kit (Vector Lab, USA) diluted in 0.01% Triton X-100/PBS. For nuclear staining, sections were incubated with 1 μg/ml Hoechst 33258 in PBS for 10 min, after rinsing the coverslips were mounted using Fluoromount (Dako, USA) and observed under a Nikon Diaphot inverted microscope, equipped for epifluorescence (Morales et al., 2011).

### Skeletal Muscle Histology.

Architecture and histology were detected by hematoxylin-eosin (H&E) stain in transverse sections of muscle (Morales et al., 2011).

### Contractile Properties.

The isometric force of isolated muscles were measured as described previously (Cabello-Verrugio et al., 2012). Briefly, optimum muscle length (Lo) and stimulation voltage were determined from micromanipulation of muscle length to produce maximum isometric twitch force. Maximum isometric tetanic force (Po) was determined from the plateau of the frequency-force relationship after successive stimulations at 1 to 200 Hz for 450 ms, with 2-minute rests between stimuli. After determination of isometric contractile properties, muscles were subjected to a 3 repeated tetanic stimulation protocol. Muscles at Lo were maximally stimulated for 450 ms once every 5 seconds. After functional testing, muscles were removed from the bath, trimmed of their tendons and any adhering non-muscle tissue, blotted once on filter paper, and weighed. Muscle mass and Lo were used to calculate specific net force (force normalized per total muscle fiber cross-sectional area (CSA), mN/mm2)(Morales et al., 2011).

### Running Test.

Mice were subjected to a running test during 15 mM at 15 m/min in a treadmill. The number of times that mice were retarded (step backs) to the first ⅓ of the moving platform was counted (Cabello-Verrugio et al., 2012).

Cell Injection into Mdx Mice.

3 month old mdx mice were exercised for 4 months and treated with 1 mg/Kg andrographolide or Vehicle. One week after the treatment, mice were anesthetized with an intramuscular injection of physiologic saline (10 ml kg⁻¹) containing ketamine (5 mg ml⁻¹) and xylazine (1 mg ml⁻¹) and then injected with approximately 5×10⁵ tendon fibroblasts into the tibialis anterior muscle via a 0.20-mm diameter needle inserted along the cranio-caudal axis of the muscle as previously described (Gargioli et al., 2008). Fibroblasts were previously labeled with 2 mMDiI according to the protocol supplied by the manufacturer (Molecular Probes). One month after the injection, mice were sacrificed for morphological analyses.

### Single Myofiber Isolation and Satellite Cells Graft.

The isolation of single myofibers were prepared essentially as described (Kelly et al., 1995, Collins 2005), briefly extensor digitorium (EDL) and soleus muscles from 6 weeks old C57-BL10 mice were dissected and digested in 0.2% (w/v) collagenase type 1 (Sigma) in DMEM (Gibco) 4 mM L-glutathatmine (Sigma) and 1% penicillin and streptomycin solution (Sigma) for 90 mM in a 37° C. water bath. After gentle muscle trituration only single and stretched myofibers were collected in DMEM. The myofibers were washed by serial transfers in 4 dishes (pre-coated with horse serum to prevent myofiber attachment) containing DMEM. The myofibers were finally collected in DMEM containing 10% FBS, 10% horse serum, 0.5% chick embryo extract and 5 ng/ml of FGF-2 (R&D) and cultured for 30 min in a 5% CO2 cell culture incubator.

Satellite cells were separated from the myofibers by physical trituration using the method of Collins et al (2005). Briefly, the isolated intact fibers were suspended in 10 ml of complete medium and triturated with a 19 G needle mounted on a 1 ml syringe. The suspension was sequentially passed through a 70 um and 40 um cell sieve (Falcon) to remove debris. The satellite cell suspension was centrifuged for 15 min at 450 RCF. The pellet was resuspended in physiologic serum (NaCl 0.9%). An aliquot was stained with Hoechst 1 ug/ml and cholera toxin sub unit B conjugated to Alexa Fluor 488 (Invitrogen) 1 ug/ml for 5 minutes, washed with PBS and incubated with trypan blue. The double stained cells that exclude the trypan blue were counted in a hemocytometer as viable cells. The concentration of cells was adjusted to 25 cell/μl. To control the purity of the isolated satellite cell, an aliquot was seeded onto Matrigel (1 mg/ml) (sigma) and cultured overnight in complete medium for 18 hours before immunocytochemistry for myogenic markers. The grafting was performed as follows, 500 satellite cells were grafted into both the TA muscles of 7 month old mdx mice in a C57-BL10 background under anesthesia using an 8 mm 30 G needle under microscopic observation.

### Statistics.

The statistical significance of the differences between the means of the experimental groups was evaluated using one-way analysis of variance (ANOVA) with a post-hoc Bonferroni multiple-comparison test (Prism 3.0, GraphPad). A difference was considered statistically significant at p value<0.05.

### EXAMPLES

### Example 1

### Effect of Andrographolide on CTGF, Fibronectin and Collagen Type III Induction by TGF-β1 In Vitro

To evaluate if andrographolide could be an anti-fibrotic factor we determine in vitro the mRNA levels of two known pro-fibrotic factors: Connective tissue growth factor (CTGF) and Transforming growth factor type beta 1 (TGF-β1) (Cabello-Verrugio et al., 2012; Morales et al., 2011). TGF-β1 induces the expression of CTGF in skeletal muscle cells (Vial et al., 2008). The FIG. 1A shows that andrographolide reduced the induction of CTGF expression in response to TGF-β1. A molecular feature of fibrotic diseases is the accumulation ECM molecules such as collagen and fibronectin, both molecules are induced by TGF-β1. FIG. 1B shows that andrographolide decreased both fibronectin and collagen type III protein levels, induced by TGF-β1 in vitro.

### Example 2

### Effect of Andrographolide on TGF-131 in Mdx Mice

Since we show that andrographolide have anti-fibrotic effects in vitro, we decide to evaluate these results in vivo. We previously showed the antifibrotic effects of andrographolide in vitro, thus we decide to evaluate the andrographolide properties in an animal model of the disease.

The pro-fibrotic cytokine TGF-β1 is augmented in mdx mice, which is related with the induction of skeletal muscle fibrosis (Andreetta et al., 2006). Therefore we evaluate if andrographolide could modulate the expression of TGF-β1 in vivo. FIG. 2A shows that andrographolide reduced the expression of TGF-β1 in mdx mice.

The canonical signaling pathway induced by TGF-β1 is through phosphorylation of smad proteins. Thus we evaluate the activity of TGF-β1 canonical signaling pathway by immunofluorescence of phosphorylated smad2 protein (p-Smad 2). FIG. 2B shows that andrographolide reduced the number of positive nuclei for phosphorylated smad2 protein. Therefore, andrographolide reduced both expression and activity of TGF-β1 in mdx mice.

### Example 3

### Effect of Andrographolide on CTGF Action In Vivo

Another pro-fibrotic cytokine overexpressed in the skeletal muscle is CTGF (Morales et al., 2011). FIG. 3A shows that andrographolide reduced CTGF expression in mdx mice. Moreover, andrographolide inhibits the pro-inflammatory effects of CTGF in vivo. Overexpression of CTGF by an adenovirus induces inflammation and fibrosis in wild type muscles (WT), showing similar features of dystrophic muscles (Morales et al., 2011). However, andrographolide inhibited these effects. FIG. 3B shows that andrographolide reduced the number of F4/80 (a macrophages specific marker) positive cells (Tidball and Villalta, 2010).

### Example 4

### Effect of Andrographolide on Dystrophic Skeletal Muscle Damage

To evaluate if andrographolide have an effect on the dystrophic phenotype of mdx mice we evaluate through Hematoxylin and Eosin staining, the histology of the tibialis anterior muscle from WT, vehicle-treated mdx and andrographolide-treated mdx mice. FIG. 4A shows that andrographolide administration prevented the increase of damaged areas observed in the muscles of dystrophic mdx mice compared to vehicle-treated mdx mice. To specifically evaluate the damage at the sarcolemma, we use the Evans Blue dye uptake protocol (Straub et al., 1997). Dystrophic muscle fibers have membrane damage, making it permeable to some colored molecules such as Evans Blue. FIG. 4B shows Evans blue dye fluorescence in tibialis anterior muscle fibers from wild type and mdx mice treated either with vehicle or andrographolide. A lower Evans blue dye uptake was observed in the muscles fibers from mdx mice treated with andrographolide, suggesting less muscle damage. Concordantly, serum CK levels (FIG. 4C) are decreased on andrographolide-treated mdx mice. The appearance of CK in blood has been generally considered to be an indirect marker of muscle damage, particularly for diagnosis of muscular dystrophy.

These results indicate that andrographolide improves the architecture of dystrophic skeletal muscles thus preventing tissue damage.

### Example 5

### Effect of Andrographolide on Fibrosis Induction in Dystrophic Skeletal Muscle

Development of fibrosis in dystrophic skeletal muscle is characterized by an increase in ECM compounds such as fibronectin and several types of collagen (Cabello-Verrugio et al., 2012). We previously found that andrographolide decreased dystrophic skeletal muscle damage, thus we decide to evaluate the impact of this botanical drug on ECM protein levels in dystrophic mdx mice. Immunofluorescence staining of mdx mice tibialis anterior muscles treated with andrographolide revealed a strongly decrease in the accumulation of collagen type I and fibronectin (FIG. 5A). Likewise, we detected that andrographolide decreased collagen I and fibronectin protein levels by western blot analysis (FIGS. 5B and 5C). Together, these results suggest that the treatment of dystrophic skeletal muscle with andrographolide decreases the development of fibrosis in dystrophic skeletal muscle. The decrease in fibronectin and collagen levels means a reduction in fibrosis and therefore a decrease in the physical barrier that impairs cell migration into the dystrophic muscle.

### Example 6

### Effect of Andrographolide in Skeletal Muscle Strength in Dystrophic Mice

We decided to evaluate if these effects had an impact on skeletal muscle physiology, since andrographolide inhibited damage and fibrosis induction in dystrophic skeletal muscle, by evaluating contractile strength in isolated muscles. Therefore, we evaluated the impact of andrographolide treatment on maximum isometric force of dystrophic tibialis anterior muscle. FIG. 6A shows a curve of net force generated from normal and mdx muscles treated with andrographolide and stimulated with frequencies ranging from 1 to 200 Hz. Under these conditions, dystrophic skeletal muscles produced a lower net force, close to 80% or less, compared to Wild Type tibialis anterior muscles in all the ranges of stimulatory frequencies evaluated. FIG. 6A also shows that muscles from mdx mice treated with andrographolide showed a significant increase in the generation of isometric force compared to Vehicle-treated mdx mice at frequencies ranging between 50 and 100 Hz. The tetanic and twitch force showed a significant increase in the tibialis anterior muscle strength in andrographolide treated mdx mice (FIGS. 6B and 6C respectively).

Given that andrographolide treatment improved muscle strength in single dystrophic isolated muscles, we ask whether andrographolide can affect the whole body muscle performance when mdx mice are defeated on a treadmill running protocol. To address this question we performed a functional test of exercise endurance through continuous exercise (De Luca et al., 2005; De Luca et al., 2003). FIG. 6D shows that mdx mice treated with andrographolide had an enhanced performance, determined by a decreased in the number of stepbacks at the treadmill.

Together, these results indicate that andrographolide not only reduce skeletal muscle damage and fibrosis but also improves skeletal muscle strength and endurance exercise.

### Example 7

### Effect of Andrographolide on Fibrosis Action In Vivo Cell Migration into the Muscle

So far we have shown that andrographolide increase skeletal muscle strength, reducing damage and fibrosis. However, Dystrophic disorders such as a DMD have genetic origins, therefore the only way to restore the gene expression is through gene and/or cell therapies. However, gene and cell therapies represents a major challenge, since muscle is the most abundant tissue in the body and more over fibrosis reduce the efficacy of these approaches. Therefore, even if current trials are successful, they are unlikely to elicit a significant benefit when extended to people at more advanced stages of the disease. Therefore, we evaluated whether the reduction of fibrosis is able to increase the efficiency of cell therapies facilitating intramuscular cell migration. Exercised mdx mice were treated with andrographolide for 3 months, and one week later, we injected tendon fibroblasts stained in red with DiI into the tibialis anterior muscle. After one month, we measured the extent of tendon fibroblast diffusion from the injection site to the muscle boundary. Immunofluorescence analysis shows those tendon fibroblasts are mainly found in non-fibrotic or less damaged regions (FIG. 7). FIG. 7 also show that DiI-labeled cell distribution is more homogenous in non-fibrotic muscles (andrographolide-treated mdx mice) than fibrotic muscles (Vehicle-treated mdx mice). These results suggest that migrating cells avoid fibrotic regions, preferring to migrate to non-fibrotic areas. Since andrographolide reduce fibrosis, Dystrophic muscles treated with this botanical drug do not show fibrotic areas, being a more homogenous tissue in comparison with non-treated dystrophic muscle that they show fibrotic patches in different areas.

These results show for the first time, that direct inhibition of muscle fibrosis environment greatly helps muscular cell migration.

### Example 8

### Effect of Andrographolide on Muscle Stem Cell Therapy in Dystrophic Muscles

We have shown that treatment with andrographolide diminishes fibrosis associated to skeletal muscle, improving muscle strength. Moreover, the reduced fibrosis environment, clearly improved the migration of fibroblast, probably due to the reduction in the physical barrier imposed by the excess of ECM compounds associated with muscle fibrosis. These results suggest that therapy using muscle precursors cells may be improved in the andrographolide-treated mice, since wild type transplanted cells could migrate far away and colonize a mayor extent of muscle, fusing with a greater number of regenerating mdx myofibers restoring on it the expression of dystrophin. To evaluate this hypothesis, freshly purified satellite cells from isolated single myofibers from Wild type (WT) mice donors were grafted in both tibialis anterior muscles of 7 month old mdx mice, pre-treated with either andrographolide or vehicle for a 3 month period under exercise protocol. The treatment with andrographolide was stopped 1 week before the transplantation leaving enough time for complete clearance of the drug, to rule out any direct effect of the drug over the transplanted cells. One month after the satellite cell transplantation, the muscle was analyzed for the presence of myofibers expressing dystrophin and collagen-I. FIG. 5A, show that the number of dystrophin positive fibers in the mdx background was increased 3 times in the muscles of the andrographolide treated mice, compared to controls, which is quantified in the FIG. 5B. The later was accompanied by a clear reduction in collagen-I content, FIG. 5A. To check the purity of the transplanted cells, an aliquot of the cells prior to the graft were plated on ECM gel for 12 hours. Then were fixed and analyzed for the expression of the muscle specific transcription factors Pax7, MyoD and Myogenin. 92% of the nuclei were positive for at least one of them, indicating the purity of the preparation (data not shown).

One possible explanation for this result is the increased migration of the transplanted cells in the andrographolide treated mice. Alternatively, it might be possibly that the host muscles have a diminished density of satellite cells, thus, the grafted cells would have no competition for the regenerating muscle fibers or instead the transplanted cells presents a better proliferation performance in the andrographolide treated mice muscles. The first possibility seems to be plausible considering the fibroblast migration data (FIG. 5), while the second hypothesis was excluded since no changes in the number of satellite cells present on isolated EDL myofibers obtained from the same transplanted mice in FIG. 5A were observed, with a mean of 12.5 satellite cells per EDL myofiber, as shown in the FIG. 5B. To test the last possibility, whether grafted cells have improved proliferation rate or survival, we purified satellite cells from the EGFP-transgenic C57BL/6 mice that constitutively expressed the EGFP transgene under the control of the chicken b-actin gene (C57BL/6-Tg(ACTbEGFP)10sba; Act-EGFP). These satellite cells were purified and grafted exactly as the experiments described above. The muscles were dissected immediately after the transplantation (Day 0) or after 2 or 15 days (Day 2 and Day 15 respectively). Genomic DNA was purified as indicated in methods. The EGFP transgene present in the grafted muscles was detected by real time qPCR in parallel with the mouse b-actin as a housekeeping gene. Since every grafted cell carries only one copy of the EGFP gene (the homozygous EGFP mice die within 2 weeks after birth) the EGFP gene detection constitutes a specific, rapid, and objective quantitation of the grafted cells. FIG. 5C, shows that in both cases the 60% of the transplanted cells die during the first 2 days, meaning that in both cases cells proliferate more rapidly, thus increasing the percentage of cells compared with Day 0 up to 3 times. Interestingly, in non-fibrotic mice (treated with andrographolide) the percentage of cells was 3 times higher compared to the fibrotic untreated mice 15 days post transplantation.

### REFERENCES

All of the references below are incorporated herein by reference.
Acharyya, S., Villalta, S. A., Bakkar, N., Bupha-Intr, T., Janssen, P. M., Carathers, M., Li, Z. W., Beg, A. A., Ghosh, S., Sahenk, Z., et al. (2007). Interplay of IKK/NF-kappaB signaling in macrophages and myofibers promotes muscle degeneration in Duchenne muscular dystrophy. The Journal of clinical investigation 117, 889-901.
Akbar, S. (2011). Andrographis paniculata: a review of pharmacological activities and clinical effects. Alternative medicine review: a journal of clinical therapeutic 16, 66-77.
Allen, D. G., and Whitehead, N. P. (2011). Duchenne muscular dystrophy—what causes the increased membrane permeability in skeletal muscle? The international journal of biochemistry & cell biology 43, 290-294.
Andreetta, F., Bernasconi, P., Baggi, F., Ferro, P., Oliva, L., Arnoldi, E., Cornelio, F., Mantegazza, R., and Confalonieri, P. (2006). Immunomodulation of TGF-beta 1 in mdx mouse inhibits connective tissue proliferation in diaphragm but increases inflammatory response: implications for antifibrotic therapy. Journal of neuroimmunology 175, 77-86.
Arechavala-Gomeza, V., Kinali, M., Feng, L., Guglieri, M., Edge, G., Main, M., Hunt, D., Lehovsky, J., Straub, V., Bushby, K., et al. (2010). Revertant fibres and dystrophin traces in Duchenne muscular dystrophy: implication for clinical trials. Neuromuscular disorders: NMD 20, 295-301.
Blau, H. M., Webster, C., and Pavlath, G. K. (1983). Defective myoblasts identified in Duchenne muscular dystrophy. Proceedings of the National Academy of Sciences of the United States of America 80, 4856-4860.
Brandan, E., Fuentes, M. E., and Andrade, W. (1992). Decorin, a chondroitin/dermatan sulfate proteoglycan is under neural control in rat skeletal muscle. Journal of neuroscience research 32, 51-59.
Cabello-Verrugio, C., Morales, M. G., Cabrera, D., Vio, C. P., and Brandan, E. (2012). Angiotensin II receptor type 1 blockade decreases CTGF/CCN2-mediated damage and fibrosis in normal and dystrophic skeletal muscles. Journal of cellular and molecular medicine 16, 752-764.
Calabrese, C., Berman, S. H., Babish, J. G., Ma, X., Shinto, L., Dorr, M., Wells, K., Wenner, C. A., and Standish, L. J. (2000). A phase I trial of andrographolide in HIV positive patients and normal volunteers. Phytotherapy research: PTR 14, 333-338.
De Luca, A., Nico, B., Liantonio, A., Didonna, M. P., Fraysse, B., Pierno, S., Burdi, R., Mangieri, D., Rolland, J. F., Camerino, C., et al. (2005). A multidisciplinary evaluation of the effectiveness of cyclosporine a in dystrophic mdx mice. The American journal of pathology 166, 477-489.
De Luca, A., Pierno, S., Liantonio, A., Cetrone, M., Camerino, C., Fraysse, B., Mirabella, M., Servidei, S., Ruegg, U. T., and Conte Camerino, D. (2003). Enhanced dystrophic progression in mdx mice by exercise and beneficial effects of taurine and insulin-like growth factor-1. The Journal of pharmacology and experimental therapeutics 304, 453-463.
Desguerre, I., Mayer, M., Leturcq, F., Barbet, J. P., Gherardi, R. K., and Christov, C. (2009). Endomysial fibrosis in Duchenne muscular dystrophy: a marker of poor outcome associated with macrophage alternative activation. Journal of neuropathology and experimental neurology 68, 762-773.
Gargioli, C., Coletta, M., De Grandis, F., Cannata, S. M., and Cossu, G. (2008). PlGF-MMP-9-expressing cells restore microcirculation and efficacy of cell therapy in aged dystrophic muscle. Nature medicine 14, 973-978.
Kapsa, R., Kornberg, A. J., and Byrne, E. (2003). Novel therapies for Duchenne muscular dystrophy. Lancet neurology 2, 299-310.
Larrain, J., Cizmeci-Smith, G., Troncoso, V., Stahl, R. C., Carey, D. J., and Brandan, E. (1997). Syndecan-1 expression is down-regulated during myoblast terminal differentiation. Modulation by growth factors and retinoic acid. The Journal of biological chemistry 272, 18418-18424.
Lee, M. J., Rao, Y. K., Chen, K., Lee, Y. C., Chung, Y. S., and Tzeng, Y. M. (2010a). Andrographolide and 14-deoxy-11,12-didehydroandrographolide from Andrographis paniculata attenuate high glucose-induced fibrosis and apoptosis in murine renal mesangeal cell lines. Journal of ethnopharmacology 132, 497-505.
Lee, T. Y., Lee, K. C., and Chang, H. H. (2010b). Modulation of the cannabinoid receptors by andrographolide attenuates hepatic apoptosis following bile duct ligation in rats with fibrosis. Apoptosis: an international journal on programmed cell death 15, 904-914.
Malik, V., Rodino-Klapac, L. R., Viollet, L., Wall, C., King, W., Al-Dahhak, R., Lewis, S., Shilling, C. J., Kota, J., Serrano-Munuera, C., et al. (2010). Gentamicin-induced readthrough of stop codons in Duchenne muscular dystrophy. Annals of neurology 67, 771-780.
Mendell, J. R., Rodino-Klapac, L., Sahenk, Z., Malik, V., Kaspar, B. K., Walker, C. M., and Clark, K. R. (2012). Gene therapy for muscular dystrophy: lessons learned and path forward. Neuroscience letters 527, 90-99.
Mendell, J. R., Rodino-Klapac, L. R., and Malik, V. (2010). Molecular therapeutic strategies targeting Duchenne muscular dystrophy. Journal of child neurology 25, 1145-1148.
Morales, M. G., Cabello-Verrugio, C., Santander, C., Cabrera, D., Goldschmeding, R., and Brandan, E. (2011). CTGF/CCN-2 over-expression can directly induce features of skeletal muscle dystrophy. The Journal of pathology 225, 490-501.
Osses, N., and Brandan, E. (2002). ECM is required for skeletal muscle differentiation independently of muscle regulatory factor expression. American journal of physiology Cell physiology 282, C383-394.
Pichavant, C., Aartsma-Rus, A., Clemens, P. R., Davies, K. E., Dickson, G., Takeda, S., Wilton, S. D., Wolff, J. A., Wooddell, C. I., Xiao, X., et al. (2011). Current status of pharmaceutical and genetic therapeutic approaches to treat DMD. Molecular therapy: the journal of the American Society of Gene Therapy 19, 830-840.
Rajagopal, S., Kumar, R. A., Deevi, D. S., Satyanarayana, C., and Raj agopalan, R. (2003). Andrographolide, a potential cancer therapeutic agent isolated from Andrographis paniculata. Journal of experimental therapeutics & oncology 3, 147-158.
Shen, Y. C., Chen, C. F., and Chiou, W. F. (2002). Andrographolide prevents oxygen radical production by human neutrophils: possible mechanism(s) involved in its anti-inflammatory effect. British journal of pharmacology 135, 399-406.
Straub, V., Rafael, J. A., Chamberlain, J. S., and Campbell, K. P. (1997). Animal models for muscular dystrophy show different patterns of sarcolemmal disruption. The Journal of cell biology 139, 375-385.
Tidball, J. G., and Villalta, S. A. (2010). Regulatory interactions between muscle and the immune system during muscle regeneration. American journal of physiology Regulatory, integrative and comparative physiology 298, R1173-1187.
Varga, J., Brenner, D. A., and Phan, S. H. (2005). Fibrosis Research: Methods and Protocols (Humana Press).
Verma, S., Anziska, Y., and Cracco, J. (2010). Review of Duchenne muscular dystrophy (DMD) for the pediatricians in the community. Clinical pediatrics 49, 1011-1017.
Vial, C., Zuniga, L. M., Cabello-Verrugio, C., Canon, P., Fadic, R., and Brandan, E. (2008). Skeletal muscle cells express the profibrotic cytokine connective tissue growth factor (CTGF/CCN2), which induces their dedifferentiation. Journal of cellular physiology 215, 410-421.
Wagner, K. R., Hamed, S., Hadley, D. W., Gropman, A. L., Burstein, A. H., Escolar, D. M., Hoffman, E. P., and Fischbeck, K. H. (2001). Gentamicin treatment of Duchenne and Becker muscular dystrophy due to nonsense mutations. Annals of neurology 49, 706-711.
Wynn, T. A. (2008). Cellular and molecular mechanisms of fibrosis. The Journal of pathology 214, 199-210.
Xia, Y. F., Ye, B. Q., Li, Y. D., Wang, J. G., He, X. J., Lin, X., Yao, X., Ma, D., Slungaard, A., Hebbel, R. P., et al. (2004). Andrographolide attenuates inflammation by inhibition of NF-kappa B activation through covalent modification of reduced cysteine 62 of p50. J Immunol 173, 4207-4217.
Zhou, L., and Lu, H. (2010). Targeting fibrosis in Duchenne muscular dystrophy. Journal of neuropathology and experimental neurology 69, 771-776.
Alderton, J. M., et al. (2000) How Calcium Influx through Calcium Leak Channels is Responsible for the Elevated Levels of Calcium-dependent Proteolysis in Dystrophic Myotubes. TCM 10(6): 268-272.
Blake, et al. (2002) Function and Genetics of Dystrophin and Dystrophin-Related Proteins in Muscle Physiol Rev. 82: 291-329
Boldrin, L., et al. (2012) Donor Satellite Cell Engraftment is Significantly Augmented when the Host Niche is Preserved and Endogenous Satellite Cells are Incapacitated. Stem Cells 30: 1971-1984.
Emery, Alan E H (2002) The Muscular Dystrophies. Lancet 359: 687-695
Sacco, P., Jones, D. A., Dick, J. R. T. Vrbova, G. (1992) Contractile properties and susceptibility to exercise-induced damage of normal and mdx mouse tibialis anterior muscle. Clinical Science 82: 227-236.

What is claimed is:

1. A method of treating a muscular dystrophy disease in a patient, the method comprising administering an effective amount of a botanical drug isolated from Andrographis paniculata in combination with cell therapy for treatment of muscular dystrophies to improve skeletal muscle performance.

2. The method of claim 1, wherein said muscular dystrophy is Duchenne muscular dystrophy (DMD).

3. The method of claim 1, wherein the botanical drug isolated from Andrographis paniculata is an andrographolide.

4. The method of claim 1, wherein the andrographolide is administered prior to cell therapy.

5. The method of claim 4, wherein the andrographolide is administered over three months.

6. The method of claim 4 wherein the treatment with andrographolide is stopped one week before the stem cell therapy.

7. The method of claim 1, wherein cell therapy for treatment of muscular dystrophies comprises transplanting skeletal muscle cells in dystrophic muscles.

8. The method of claim 1, wherein the andrographolide diminishes fibrosis associated with skeletal muscle, reduces fibrosis environment, improves muscle strength and migration of fibroblast in dystrophic muscles.

9. The method of claim 1, wherein the andrographolide improves transplanted cell proliferation in cell therapy.

10. The method of claim 1, wherein the andrographolide improves skeletal muscle strength and physiology.
